(19) United States
(12) Patent Application Publication
(10) Pub. No.: US 2022/0347085 A1
(43) Pub. Date: Nov. 3, 2022

(54) REPELLENT COMPOSITION AND USES

(71) Applicants: Université Bourgogne, DIJON CEDEX (FR); Institut national de recherche pour l'agriculture l'alimentation et l'environnement, PARIS (FR); Centre National de la Recherche Scientifique, PARIS (FR)

(72) Inventors: Martine BERTHELOT-GROSJEAN, SAINT-SAUVEUR (FR); Yaël GROSJEAN, SAINT-SAUVEUR (FR); Gérard MANIERE, BELLEFOND (FR)

(21) Appl. No.: 17/642,634

(22) PCT Filed: Sep. 10, 2020
(86) PCT No.: PCT/EP2020/075386

§ 371 Date: Mar. 11, 2022

(30) Foreign Application Priority Data
Sep. 13, 2019 (EP) 19306102.5

(51) Int. Cl. A61K 8/9794; A61K 8/64; A61K 8/42; A61K 8/73; A61Q 19/08; A61K 8/31

(52) U.S. Cl. CPC A61K 8/9794; A61K 8/64; A61K 8/42; A61K 8/735; A61Q 19/08; A61K 8/31

(57) ABSTRACT

The present invention relates to the use of at least one fatty acid, advantageously volatile and odorous, selected from the group comprising propionic acid, butyric acid and/or a derivative thereof as a repellent active principle and/or for controlling the reproduction of brachycera, and to the use of an, in particular phytosanitary, composition comprising at least one fatty acid, advantageously volatile and odorous, selected from the group comprising propionic acid, butyric acid and/or a derivative thereof as a repellent active principle and/or for controlling the reproduction of brachycera advantageously by olfaction, and non-insecticidal. The present invention can be used in the agricultural, veterinary and phytosanitary fields.

### TECHNICAL FIELD

The present invention relates to the use of at least one fatty acid selected from the group comprising propionic acid, butyric acid and/or a derivative thereof as a repellent active principle and/or for controlling the reproduction of brachycera.

The present invention also relates to the use of at least one fatty acid, in particular volatile, selected from the group comprising propionic acid and butyric acid as a repellent active principle and/or for controlling the reproduction of brachycera.

The present invention also relates to the use of an, in particular phytosanitary, composition comprising at least one fatty acid selected from the group comprising propionic acid, butyric acid and/or a derivative thereof as a repellent active principle and/or for controlling the reproduction of brachycera.

The present invention also relates to the use of an, in particular phytosanitary, composition comprising at least one fatty acid selected from the group comprising propionic acid, butyric acid and/or a derivative thereof as a repellent active principle and/or for controlling the reproduction of brachycera.

The present invention can be used in particular in the agricultural, veterinary and phytosanitary fields.

In the description below, the references between square brackets ([ ]) refer to the list of references set out at the end of the text.

### Prior Art

In the field of agriculture, numerous insects are considered as insect pests. These are in particular insects which cause and/or are the source of crop damage and/or spoilage.

All agricultural crops, for example cereal, fruit or vegetable crops, may be affected by insect pests. In a general manner, the insect pests are specific to one type of crop and/or plant, and may affect the crops and/or plants differently. In other words, it is often the case that the insect pests are specific to one crop/plant, and that the effect on the crop/plant is specific.

For example, the insect pests may have an effect on the fruit of the plant, for example by eating it, and/or may affect the “health” of the plant, for example by carrying pathologies, for example viruses, fungi, etc., which may spoil the growth and/or the fruit/flower production etc. of the plant/crop.

All the parts of the plant/crop may be affected by insect pests. For example, the insect pests may spoil the leaves, the stems, the buds, the bark, the wood, the roots, the flowers, the fruits, the seeds, at all stages of their development. Furthermore, the insect pests may also affect the products/fruits and/or any other element obtained from harvests.

There is therefore a real need, in the prior art, to protect and/or prevent attack and/or spoilage of plants/crops by insect pests. There is also a real need to protect and/or prevent attack and/or spoilage of harvests by insect pests, without, however, endangering the ecosystem and the biodiversity. There is also an urgent need to find alternatives to insecticides which destroy insect species in an uncontrolled manner.

In order to preserve the plants/crops against insect pests, methods and/or products are known and used. For example, alternation from one year to the next, and/or agricultural plots of cultivated plants, and/or a selection of particular plants, is used in order to reduce the effects of insect pests. However, the protection and/or the effects are limited. Furthermore, selecting resistant plants may lead to a genetic impoverishment of indigenous plants, and/or to a reduction in biodiversity. Mechanical methods are also used. Chemical methods are also used, in particular the use of synthetic and/or chemical pesticides and insecticides, of repellent compositions, etc. However, these methods have a “non-selective” effect, targeting all insects indiscriminately, and may thus have harmful effects on auxiliary and/or essential insects such as honeybees and/or bumblebees. Furthermore, the chemical methods are currently controversial, in particular because they can affect human and/or animal health, and/or be the cause of soil pollution, water course pollution, etc. Moreover, the insecticides used may be absorbed by the plants. Thus, the plants and/or the crops produced may have concentrations of these compositions that are incompatible with animal and/or human consumption.

There is therefore a real need to find a novel means and/or a novel composition that overcomes these failings, disadvantages and obstacles of the prior art, in particular a method and/or a composition which makes it possible to preserve the plants and/or the crops from pests, while conserving and/or preserving the ecosystem, the biodiversity, and/or human health and/or animal health.

### DESCRIPTION OF THE INVENTION

Specifically, the present invention aims to meet these needs and overcome these disadvantages by providing at least one fatty acid selected from the group comprising propionic acid, butyric acid and/or a derivative thereof for use as a repellent active principle and/or for controlling the reproduction of brachycera.

The inventors have surprisingly demonstrated that non-insecticidal compositions, i.e. volatile fatty acids according to the invention, advantageously have a repellent effect with respect to brachycera.

Indeed, the inventors have surprisingly and unexpectedly demonstrated that the present invention, in particular the use of volatile fatty acids according to the invention, has a repellent effect with respect to brachycera, advantageously by way of olfactory perception, in particular by olfactory perception of adults.

The inventors have also surprisingly and unexpectedly demonstrated that the present invention, in particular the use of volatile fatty acids according to the invention, has an effect, in particular on the behavior of brachycera associated with their reproduction.

Furthermore, the inventors are the first to have demonstrated, surprisingly and unexpectedly, that the use according to the invention, in particular the use of fatty acids according to the invention, has an effect on the behavior of insect pests, in particular by way of the olfactory perception of said molecules.

Moreover, the inventors are the first to have demonstrated, surprisingly and unexpectedly, that the use according to the invention, in particular the use of, in particular volatile, fatty acids according to the invention, has an effect on the behavior of pests, preferably adult insects, in particular by way of the olfactory perception of said molecules, without affecting the fatality of the insect pests. In other words, the inventors have demonstrated that the present invention has no insecticidal effect on the insect pests.

In particular, the inventors have surprisingly demonstrated that the use of the fatty acids according to the invention has a repellent effect, makes it possible to reduce or even eliminate the courtship of insects, and may bring about anesthesia of the insects.

Moreover, the inventors have advantageously demonstrated that the use according to the invention, in particular the use of, in particular volatile, fatty acids according to the invention, may bring about a reversible anesthesia of the brachycera, advantageously making it possible, in contrast with known insecticides, to be non-toxic with respect to the brachycera. In other words, the inventors have shown that the present invention may advantageously bring about a reversible anesthesia corresponding to temporary anesthesia which does not bring about any toxicity in the adult brachycera.

The inventors have also surprisingly demonstrated that the present invention, for example by way of reducing the courtship, advantageously makes it possible to reduce and/or prevent the proliferation of insect pests.

The inventors have also demonstrated that the present invention advantageously makes it possible to reduce and/or prevent the spread of insect pests, without affecting their role in the ecosystem outside of the crops to be protected.

The inventors have also surprisingly demonstrated that the present invention advantageously makes it possible to reduce and/or prevent the reproduction and/or copulation of pests.

Furthermore, the inventors have surprisingly demonstrated that the present invention has an effect on the insect pests by way of olfactory perception, and advantageously makes it possible to reduce and/or suppress the detection, by insect pests, of attractive olfactory signals, which for example cause and/or are capable of stimulating the courtship preceding the mating of insects. In other words, the inventors have surprisingly demonstrated that the present invention advantageously makes it possible to alter the detection, by insect pests, of attractive olfactory signals, for example between male and female.

In particular, the inventors have surprisingly shown that the present invention, by way of the olfactory perception, advantageously makes it possible to reduce and/or inhibit courtship and copulation of insect pests, behaviors prior to said insect pests selectively laying eggs, for example on plants and/or crops, which for example the offspring eat, without killing these insects, and thus protects their importance for the outside ecosystem, in particular plants and/or crops to be protected.

Moreover, the invention uses fatty acids and/or derivatives thereof which are compounds and/or molecules that are non-toxic for the environment, ecosystem-friendly, and without known harmful effects for human and animal health.

The present invention relates to the use of at least one fatty acid selected from the group comprising propionic acid, butyric acid and/or a derivative thereof as a repellent active principle and/or for controlling the reproduction of brachycera.

The present invention also relates to the use of at least one, advantageously volatile, fatty acid selected from the group comprising propionic acid and butyric acid as a repellent active principle and/or for controlling the reproduction of brachycera.

The present invention also relates to the use of at least two fatty acids selected from the group comprising propionic acid, butyric acid and/or a derivative thereof as repellent active principles and/or for controlling the reproduction of brachycera.

The present invention also relates to the use of propionic acid and butyric acid as, advantageously volatile, repellent active principles and/or for controlling the reproduction of brachycera.

In the present document, the term “derivative” denotes a chemical compound or a molecule produced from a parent compound by way of one or more chemical reactions.

In the present invention, the term “salt” denotes salts that are suitable for human or veterinary pharmaceutical use without toxicity, irritation, allergic reaction, or other harmful effect which is inappropriate for a medical use, and that have a reasonable balance of benefits and risks.

In the present invention, the term “salt” also denotes salts that are suitable for phytosanitary use without toxicity, irritation, allergic reaction, or other harmful effect which is inappropriate for a use for example in agriculture.

In the present document, the term “repellent” means distancing and/or pushing away the brachycera from plants and/or vegetables and/or fruits, and/or all or some of the plants and/or vegetables and/or mammals. The term “repellent” also means inhibiting the attraction of the brachycera to the plants and/or vegetables and/or fruits, and/or all or some of the plants and/or vegetables and/or mammals. The term “repellent” also means a lack of interest of brachycera for an environment and/or space in which one or more volatile odorous molecule(s) is (are) present.

In the present document, the term “control of the reproduction” means an inhibition and/or a stabilization of the reproduction of brachycera, in particular by inhibition of the courtship and/or reduction of the courtship, and/or inhibition of flights in swarms and/or inhibition of offering, by the male, of prey to the female, and/or inhibition of the creation of a cocoon by the male. In other words, “control of the reproduction” means a reduction and/or inhibition of the courtship and/or copulation of brachycera, which are behaviors, preferably adult, prior to said brachycera laying eggs, preferably selectively, in particular prior to laying eggs for example on plants and/or vegetables which the offspring, for example the larvae, preferably eat.

In the present document, brachycera mean the insects belonging to the family of the muscidae, the oestridae, the hippoboscidae, the nycteribiidae, the syrphidae, the tachinidae, the thaumatoxena, the thermitoxena. Preferably, brachycera mean the insects belonging to the family of the oestridae, the hippoboscidae, the nycteribiidae, the syrphidae, the tachinidae, the thaumatoxena, the thermitoxena. For example, these may be insects selected from the group comprising the insects of the genus Musca, Drosophila, Glossina, Calliphora, Auchmeromyia, Lucilia, Sarcophaga, Stomoxys; preferably these may be insects selected from the group comprising the insects of the genus Drosophila, Glossina, Calliphora, Auchmeromyia, Lucilia, Sarcophaga, Stomoxys. These may for example be insects belonging to the genus Drosophila selected from the group comprising Drosophila suzukii, Drosophila melanogaster, preferably Drosophila suzukii.

In the present document, when the insects are of the genus Musca, they may be any insect of the genus Musca, with the exception of Musca domestica.

In the present document, the brachycera also include the insects belonging to the family of the trypetidae, also referred to as tephritidae. These may for example be insects selected from the group comprising insects of the genus Bactrocera. These may for example be insects selected from the group comprising Bactrocera oleae.

In the present document, brachycera may be selected from the insects belonging to the family of the oestridae, the hippoboscidae, the nycteribiidae, the syrphidae, the tachinidae, the thaumatoxena, the thermitoxena and/or trypetidae.

In the present document, the brachycera may be selected from the group comprising Drosophila suzukii and Bactrocera oleae.

In the present document, “propionic acid,” also referred to as propanoic, means a volatile C3 fatty acid of empirical formula C₃H₆O₂, and/or of semi-structural formula (I) as follows:

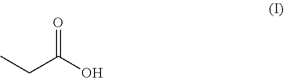

Advantageously, the propionic acid is a volatile fatty acid.

Advantageously, according to the invention, the propionic acid is odorous.

In the present document, an odorous compound means a compound which gives off an odor.

In the present document, derivative of propionic acid means any derivative and/or salts known to a person skilled in the art. This may for example be a derivative selected from the group comprising a propionic acid ester, a propionic acid amine, and/or a mixture thereof.

According to the invention, the propionic acid ester may be any propionic acid ester known to a person skilled in the art. This may for example be an ester of formula CH₃—CH₂—COOR₁ (III), in which R₁ may be a C₁ to C₆ alkyl, for example a methyl, an ethyl, a butyl, a propyl, a pentyl, a hexyl, an R₁₁R₁₂ group in which R₁₁ is an anion and R₁₂ is a cation selected from the group of the alkali metals comprising lithium, sodium, potassium, rubidium, and cesium. For example, the propionic acid ester may be selected from the group comprising ethyl propanoate, propyl propanoate, butyl propanoate, isoamyl propanoate, isobutyl propanoate, and/or isopropyl propanoate. The propionic acid ester is preferably ethyl propanoate.

According to the invention, the propionic acid amine may be any propionic acid amine known to a person skilled in the art. For example, the propionic acid may be selected from the group comprising propylamine, dipropylamine, tripropylamine, isopropylamine, diisopropylamine, triisopropylamine.

Advantageously, when the ester is a derivative of propionic acid, preferably ethyl propanoate or ethyl butyrate, it exhibits a fragrance which is more agreeable, to humans, than the acid.

According to the invention, the propionic acid and/or the derivative thereof may be at a concentration of between 0.1 and 30% by volume (v/v). According to the invention, the concentration of the propionic acid and/or a derivative thereof may be adjusted depending on the brachycerus. For example, the propionic acid and/or the derivative thereof may be at a concentration of between 0.5% and 1%, 1% and 2%, or 2% and 30% by volume (v/v). According to the invention, the concentration of the propanoic acid may be equal to 0.5%, 1%, 2%, 4%, 5% by volume (v/v). According to the invention, the concentration of the propionic acid derivative may be between 2% and 30% by volume (v/v). For example, the concentration of ethyl propanoate may be 10% by volume (v/v).

In the present document, “butyric acid,” also referred to as butanoic acid, means a volatile C4 fatty acid of empirical formula C₄H₈O₂, and/or of semi-structural formula (II) as follows:

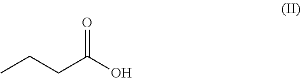

Advantageously, the butyric acid is a volatile fatty acid.

Advantageously, according to the invention, the butyric acid is odorous.

In the present document, derivative of butyric acid means any derivative and/or salts known to a person skilled in the art. This may for example be a derivative selected from the group comprising a butyric acid ester and/or a salt thereof.

According to the invention, the butyric acid ester may be any butyric acid ester known to a person skilled in the art. This may for example be an ester of formula CH₃—(CH₂)₂—COOR₂ (III), in which R₂ may be a C₁ to C₆ alkyl, for example a methyl, an ethyl, a butyl, a propyl, a pentyl, a hexyl, an R₁₁R₁₂ group in which R₁₁ is an anion and Rig is a cation selected from the group of the alkali metals comprising lithium, sodium, potassium, rubidium, and cesium. For example, the butyric acid ester may be selected from the group comprising ethyl butyrate, propyl butyrate, butyl butyrate, isoamyl butyrate, isobutyl butyrate, and/or isopropyl butyrate.

Advantageously, when it is a butyric acid ester, preferably ethyl butyrate, it has a fragrance which is agreeable to humans (pineapple).

According to the invention, the butyric acid and/or the derivative thereof may be at a concentration of between 0.1 and 3% by volume (v/v).

According to the invention, the concentration of the butyric acid and/or the derivative thereof may be adjusted depending on the brachycerus. For example, the butyric acid and/or the derivative thereof may be at a concentration of between 0.5% and 3% by volume (v/v). According to the invention, the concentration of the butyric acid may be equal to 0.5%, 1%, 2% by volume (v/v). According to the invention, the concentration of the butyric acid derivative may be advantageously equal to 2% by volume (v/v). According to the invention, the concentration of the butyric acid derivative may be between 2% and 3% by volume (v/v). For example, when the derivative is ethyl butyrate, the concentration may be greater than or equal to 2% by volume (v/v).

According to the invention, when propionic acid and/or a derivative thereof, and butyric acid and/or a derivative thereof, are used as active principles, the concentration of propionic acid and/or of the derivative may be between 0.1 and 0.5% by volume (v/v), and the concentration of butyric acid and/or of the derivative may be between 0.1% and 0.5% by volume (v/v).

The present invention also relates to a composition comprising at least one fatty acid selected from the group comprising propionic acid, butyric acid and/or a derivative thereof, and an acceptable support.

The present invention also relates to a composition comprising at least one, advantageously volatile, fatty acid selected from the group comprising propionic acid, butyric acid and/or a derivative thereof, and an acceptable support.

The present invention also relates to a composition comprising at least one, advantageously volatile, fatty acid selected from the group comprising propionic acid, butyric acid, and an acceptable support.

The present invention also relates to a composition comprising propionic acid and butyric acid and an acceptable support.

The present invention also relates to a composition comprising propionic acid and butyric acid.

The present invention also relates to the use of a composition comprising at least one fatty acid selected from the group comprising propionic acid, butyric acid and/or a derivative thereof, and an acceptable support, as a repellent and/or in order to control the reproduction of brachycera.

The present invention also relates to the use of a composition comprising at least one, advantageously volatile, fatty acid selected from the group comprising propionic acid, butyric acid and/or a derivative thereof, and an acceptable support, as a repellent and/or in order to control the reproduction of brachycera.

The present invention also relates to the use of a composition comprising propionic acid, butyric acid and an acceptable support, as a repellent and/or in order to control the reproduction of brachycera.

The present invention also relates to the use of a composition comprising propionic acid and butyric acid, as a repellent and/or in order to control the reproduction of brachycera.

According to the invention, the support may be any support that is known to a person skilled in the art and is suitable to be mixed and/or associated with fatty acids in a composition. This may for example be an oil, for example a mineral oil, a vegetable oil, and/or a mixture thereof. It may, for example, be any mineral oil that is known to a person skilled in the art and is suitable for use in the pharmaceutical, cosmetic, and/or phytosanitary field. It may for example be a mineral oil selected from the group comprising paraffin oil or Vaseline. The mineral oil is preferably paraffin oil. Advantageously, the paraffin oil has no significant effect with respect to the brachycera. Furthermore, the paraffin oil advantageously allows for a constant release of the fatty acid(s) and/or derivative(s) with which it is mixed.

Advantageously, when the support is paraffin oil, it makes it possible to keep the fatty acids in non-ionic form (COOH) in the composition.

According to the invention, the vegetable oil may be any vegetable oil that is known to a person skilled in the art and is suitable for use in the pharmaceutical, cosmetic, and/or phytosanitary field.

According to the invention, the composition may be a cosmetic or dermatological composition, a pharmaceutical composition, and/or a phytosanitary composition.

In the present document, “cosmetic composition” means any form of cosmetic composition that is known to a person skilled in the art. The cosmetic composition according to the invention may comprise one or more adjuvants and/or cosmetically acceptable support known to a person skilled in the art. Except in the case where an adjuvant and/or cosmetically acceptable support were to prove incompatible with the compounds according to the invention, for example producing any kind of undesirable biological effect, and/or had an odor and/or an effect which could mask the odor of the compounds according to the invention, or indeed interacting in a harmful manner with any other component of the cosmetic composition, the use thereof is considered to come under the scope of the present invention. It may for example be one or more adjuvant(s) selected from agents of the ester type, hydrating agents, softening agents, emulsifiers, surfactants, mineral thickening agents, organic thickening agents, associative or otherwise, hydrosoluble and liposoluble organic solar filters, mineral solar filters, silicon compounds, scents, preservatives, ceramides and pseudo-ceramides, vitamins and provitamins, proteins, sequestrating agents, alkalizing agents, acidifying agents, reducing agents, oxidizing agents, mineral fillers, colorants, or any other suitable adjuvant which may be cited in the INCI (International Nomenclature of Cosmetic Ingredients) dictionary published by the PCPC (Personal Care Products Council).

According to the invention, the cosmetic or dermatological composition may, for example, be in any form known to a person skilled in the art which can be used in the cosmetic field. Except in the case where the formulation of the cosmetic or dermatological composition were incompatible with the compounds according to the invention, for example producing any kind of undesirable biological effect, and/or an odor and/or an effect which could mask the odor of the compounds according to the invention, or interacting in a harmful manner with any other component of the cosmetic composition, the use thereof is considered to come under the scope of the present invention. From his general knowledge, a person skilled in the art would be able to adapt the formulation depending on the intended application.

In the present document, the cosmetic composition may be obtained by any appropriate method known to a person skilled in the art for preparing a cosmetic composition. This may be a mixture of surface-active materials in water.

In the present document, “pharmaceutical composition” means any form of suitable pharmaceutical composition that is known to a person skilled in the art. In the present document, the pharmaceutical composition may for example be a topical solution, a galenic orodispersible form, and/or an aerodispersible solution, for example a liquid solution, or a spray.

According to the present invention, the pharmaceutical composition may be a pharmaceutical composition for topical or transdermal administration, for example selected from the group comprising ointments, creams, gels, lotions, patches, and mousses. Except in the case where the formulation of the pharmaceutical composition would be incompatible with the compounds according to the invention, for example producing any kind of undesirable biological effect, and/or an odor and/or an effect which could mask the odor of the compounds according to the invention, or interacting in a harmful manner with any other component of the pharmaceutical composition, the use thereof is considered to come under the scope of the present invention. From his general knowledge, a person skilled in the art would be able to adapt the formulation depending on the intended application.

In the present, the composition may be formulated and/or adapted according to the administration thereof. The composition of the present invention may also comprise at least one other active ingredient, in particular another therapeutically active ingredient, for example for simultaneous or separate use, or use spread over time, according to the galenic formulation used.

As described above, the pharmaceutically acceptable compositions of the present invention may further comprise a pharmaceutically acceptable support, an adjuvant, or a vehicle which, as defined in the present document, includes any solvent, diluent or other liquid vehicle, dispersion or suspension aid, surface-active agent, isotonic agent, thickening or emulsifying agent, preservative, solid binder, lubricant, and the like, that is suitable for the particular dosage form desired. Remington Pharmaceutical Sciences, sixteenth edition, E. W. Martin (Mack Publishing Co., Easton, Pa., 1980 [5]) describes different supports used in the formulation of pharmaceutically acceptable compositions, and known techniques for the preparation thereof. Except in the case where a conventional support medium were to prove incompatible with the compounds according to the invention, for example producing any kind of undesirable biological effect, and/or had an odor which could mask the odor of the compounds according to the invention, or indeed interacting in a harmful manner with any other component of the pharmaceutically acceptable composition, the use thereof is considered to come under the scope of the present invention. Some examples of materials which may serve as pharmaceutically acceptable supports include, but are not limited to, ion exchangers, buffer substances, water, salts or wax electrolytes, sugars; starches, excipients, oils, glycols, esters, as well as other compatible non-toxic lubricants, as well as coloring agents, mold release agents, coating agents, sweetening agents, aromas, preservatives, and antioxidants may also be present in the composition, according to the judgment of the galenic form specialist.

According to the invention, during the use thereof, the fatty acid selected from the group comprising propionic acid, butyric acid and/or a derivative thereof, and/or the composition comprising at least one fatty acid selected from the group comprising propionic acid, butyric acid and/or a derivative thereof, may be applied by any suitable means known to a person skilled in the art. For example, it may be an application by means of a diffuser, a spray, an atomizer, an impregnated support.

According to the invention, the application means can be adjusted depending on the composition used. From his general knowledge, a person skilled in the art will be able to adapt the application means to the form of the composition used.

The present invention also relates to a method for protecting at least one biotic and/or abiotic surface from brachycera, comprising application, onto said surface, of at least one fatty acid selected from the group comprising propionic acid, butyric acid and/or a derivative thereof, or a composition comprising at least one fatty acid selected from the group comprising propionic acid, butyric acid and/or a derivative thereof. The present invention also relates to an ex-vivo method for protecting at least one biotic and/or abiotic surface from brachycera, comprising application, onto said surface, of at least one fatty acid selected from the group comprising propionic add, butyric acid or a composition comprising propionic acid and/or butyric acid.

The present invention also relates to the ex-vivo use of propionic acid and of butyric acid and/or of a composition comprising propionic acid and butyric acid for protecting at least one biotic and/or abiotic surface from brachycera.

The propionic add and/or a derivative thereof is as defined above.

The butyric add and/or a derivative thereof is as defined above.

The composition is as defined above.

By protection of a surface, the present document means the absence of egg-laying on the surface due in particular to the reduction and/or inhibition of the courtship and/or copulation of brachycera, which are behaviors prior to said brachycera laying eggs, and/or inhibition of the attractiveness and/or a disinterest of brachycera for a surface that is present in the environment and/or space in which one or more volatile odorous molecule(s) is (are) present.

In the present, surface means any surface known to a person skilled in the art. It may for example be a biotic or abiotic surface.

In the present, biotic surface means any biotic surface known to a person skilled in the art. It may for example be a biotic surface selected from the group comprising the skin, the mucous membranes, the hair, the skin appendages, the eyes, the feathers.

In the present, the biotic surface may be a healthy surface and/or a surface exhibiting at least one lesion and/or an infection. This may for example be a biotic surface comprising a wound, an infected wound, a wound that is scarring, a sutured wound, a scar, a burn.

In the present, the biotic surface may be a surface of all or part of a vegetable and/or plant. It may for example be the surface of leaves, fruits, flowers, petals, pistils, stems, branches, trunks. Preferably, it can be the surface of fruits.

In the present, abiotic surface means any abiotic surface known to a person skilled in the art. It may also be any device surface known to a person skilled in the art. It may for example be the surface of a medical device, any surface present in medical emergency rooms, medical treatment rooms, operating theaters, operating blocks, intensive care unit (ITU) rooms, infectious diseases unit rooms. It may also be any surface of laboratory rooms, of a biological laboratory, of a biological analysis laboratory, of an incubation room, and of another enclosed volume. It may for example be the surface of a building, for example for residential, storage and/or agricultural purposes. It may for example be the surface of roofs, walls, windows, gutters.

In the present, the abiotic surface may be formed by any material known to a person skilled in the art. It may for example be a biocompatible material or otherwise. In the present, the abiotic surface may for example be a metal surface, a surface formed by an alloy, a polymer surface. For example, the metal may be any metal known to a person skilled in the art, for example a metal selected from the group comprising titanium, copper, iron, aluminum, nickel, tungsten, silver, gold, palladium, vanadium, molybdenum. For example, the alloy may be any alloy known to a person skilled in the art, for example an alloy selected from the group comprising steel, brass, copper-nickel, copper-palladium, silver-gold, silver-palladium, molybdenum-vanadium, molybdenum-tungsten. For example, the polymer may be any polymer known to a person skilled in the art which can form and/or cover a surface. It may for example be a polymer selected from the group comprising polytetrafluoroethylene (PTFE), the polysiloxanes, the polyurethanes, the functionalized polymers.

According to the invention, the application may be carried out by any means known to a person skilled in the art. For example, the application may be carried out using a diffuser, an atomizer, a pressurized diffuser. From his general knowledge, a person skilled in the art will be able to adapt and/or select the application means depending on the surface.

Advantageously, when the fatty acids according to the invention and/or the composition according to the invention are/is applied to an abiotic surface, they make it possible to prevent the appearance of brachycera close to said surfaces, in particular they make it possible to push away and/or distance the brachycera from said surfaces.

The present invention also relates to a kit or a case containing at least one fatty acid selected from propionic acid and/or a derivative thereof, butyric acid and/or a derivative thereof, and/or a composition comprising at least one fatty acid selected from propionic acid and/or a derivative thereof, butyric acid and/or a derivative thereof, and a support comprising usage instructions.

The propionic acid and/or a derivative thereof is as defined above.

The butyric acid and/or a derivative thereof is as defined above.

The composition is as defined above.

In the present document and in the examples below, the concentrations of propionic acid and/or butyric acid and/or derivative thereof are expressed in percentages, unless otherwise stated, i.e. in volume of propionic acid and/or butyric acid and/or derivative thereof with respect to the total volume of the composition (v/v).

Other advantages may become apparent to the person skilled in the art when reading the examples below, illustrated by the figures given for illustrative purposes.

### BRIEF DESCRIPTION OF THE FIGURES

FIG. 1 is a bar chart showing the measurement of the proportion, in percentage, of courtship of male Drosophila suzukii towards females (ordinate), in the absence or in the presence of a composition comprising 0.1%, 0.5% and 1% propionic acid (abscissa). In the figure, (*), (**), (***) and (****) denote a statistical difference between the different conditions exhibiting variable quantities of propionic acid and calculated by performing a Kruskal-Wallis test followed by a post-hoc Dunn's Multiple Comparison Test.

FIG. 2 is a bar chart showing the measurement of the proportion, in percentage, of courtship of male Drosophila suzukii towards females (ordinate), in the absence or in the presence of a composition comprising 0.1%, 0.5% and 1% butyric acid (abscissa). In the figure, (*), (**), (***) and (****) denote a statistical difference between the different conditions exhibiting variable quantities of butyric acid and calculated by performing a Kruskal-Wallis test followed by a post-hoc Dunn's Multiple Comparison Test.

FIG. 3 is a bar chart showing the measurement of the proportion, in percentage, of courtship of male Drosophila suzukii towards females (ordinate), in the absence or in the presence of a composition comprising, respectively, 0.1%, 0.5% and 1% propionic acid or 0.1%, 0.5% and 1% butyric acid or mixtures corresponding to 0.1%, 0.25% or 0.5% propionic acid and butyric acid (abscissa). In the figure, (*) denotes a statistical difference between the different conditions exhibiting variable quantities of fatty acids. The statistical test performed is a Kruskal-Wallis test followed by a post-hoc Dunn's Multiple Comparison Test.

FIG. 4 is a bar chart showing the measurement of the proportion, in percentage, of copulation of Drosophila suzukii (ordinate), in the absence or in the presence of a composition comprising, respectively, 0.1%, 0.5% and 1% propionic acid (abscissa). In the figure, (*) denotes a statistical difference between the different conditions exhibiting variable quantities of propionic acid. The statistical test performed is a Kruskal-Wallis test followed by a post-hoc Dunn's Multiple Comparison Test.

FIG. 5 is a bar chart showing the measurement of the proportion, in percentage, of copulation of Drosophila suzukii (ordinate), in the absence or in the presence of a composition comprising, respectively, 0.1%, 0.5% and 1% butyric acid (abscissa). In the figure, C) denotes a statistical difference between the different conditions exhibiting variable quantities of butyric acid. The statistical test performed is a Kruskal-Wallis test followed by a post-hoc Dunn's Multiple Comparison Test.

FIG. 6 is a bar chart showing the time elapsed leading to the anesthesia of male or female Drosophila suzukii (abscissa), in minutes and in seconds (ordinate) in the presence of a composition comprising 2% propionic acid. A Mann-Whitney (nonparametric t-test) does not reveal the difference associated with the time leading to the anesthesia of flies depending on their sex.

FIG. 7 is a bar chart showing the time elapsed leading to the anesthesia of male or female Drosophila suzukii (abscissa), in minutes and in seconds (ordinate), in the presence of a composition comprising 2% butyric acid. A Mann-Whitney (nonparametric t-test) does not reveal the difference associated with the time leading to the anesthesia of flies depending on their sex.

FIG. 8 is a photograph showing a portion of the inside of a transparent test box of 1250 cm³, in which 2% propionic acid has been deposited in a petri dish covered by a gauze (outside the scope of the image shown). The black dots on the figure correspond to Drosophila suzukii flies. The arrow indicates a Drosophila suzukii fly on its back, in the anesthetized position.

FIG. 9 is a photograph showing a portion of the inside of a transparent test box of 1250 cm³, in which 2% butyric acid has been deposited in a petri dish covered by a gauze (outside the scope of the image shown). The black dots on the figure correspond to Drosophila suzukii flies. The arrow indicates a Drosophila suzukii fly on is back, in the anesthetized position.

FIG. 10 is a bar chart showing the measurement of the proportion, in percentage, of courtship of Drosophila melanogaster (ordinate), in the absence or in the presence of a composition comprising, respectively, 0.5% and 1% propionic acid (abscissa). In the figure, (*) denotes a statistical difference between the different conditions exhibiting variable quantities of propionic acid and calculated by performing a Mann-Whitney test (nonparametric test).

FIG. 11 is a bar chart showing the measurement of the proportion, in percentage, of courtship of Drosophila melanogaster (ordinate), in the absence or in the presence of a composition comprising, respectively, 0.5% and 1% butyric acid (abscissa). In the figure, (***) denotes a statistical difference between the different conditions exhibiting variable quantities of butyric acid and calculated by performing a Mann-Whitney test (nonparametric test).

FIG. 12 is a bar chart showing the time elapsed leading to the anesthesia of male or female Drosophila melanogaster (abscissa), in minutes and in seconds (ordinate), in the presence of a composition comprising 2% propionic acid. In the figure, (****) denotes a statistical difference between the different conditions exhibiting variable quantities of propionic acid and calculated by performing a Mann-Whitney test (nonparametric test).

FIG. 13 is a bar chart showing the time elapsed leading to the anesthesia of male or female Drosophila melanogaster (abscissa), in minutes and in seconds (ordinate), in the presence of a composition comprising 2% butyric acid. In the figure, (***) denotes a statistical difference between the different conditions exhibiting variable quantities of propionic acid and calculated by performing a Mann-Whitney test (nonparametric test).

FIG. 14 corresponds to three photographs of the same test box (in its entirety in FIG. 14A, and in a partial view from below (FIG. 1413 and FIG. 140)) used for studying the repellent and/or distance anesthesia behavior of a melanogaster and D. suzukii. In this transparent box, of volume 1250 cm³, 2 Petri dishes are deposited: one, covered with a gauze (visible on the right-hand side of the image showing the entirety of the test box), containing 2%, 6% or 20% propionic acid, the other containing synthetic food (visible on the left-hand side of the image). The black dots on the figure correspond to Drosophila melanogasfer flies.

FIG. 15 is a diagram showing one of six cylindrical alveolae of the behavior device used for studying the courtship of D. melanogaster. A filter paper soaked in an odor is deposited at the base of the cylinder, the central part of which paper holds a netting on which the flies tested move, and this cylinder is finally closed. For the tests of the courtship of D. melanogaster, a red light illuminates the device.

FIG. 16 is a diagram showing a behavior device used for studying the courtship of D. suzukii. It is similar to that used for D. melanogaster, but is illuminated by white light.

FIG. 17 is a bar chart showing the time elapsed leading to the anesthesia of male or female Drosophila suzukii in minutes and in seconds (ordinate) in the presence of a composition comprising 2% butyric acid, 2% propionic acid, 5% ethyl propanoate, 10% ethyl propanoate, or 30% ethyl propanoate (abscissa).

FIG. 18 is a bar chart showing the measurement of the proportion, in percentage, of courtship, of males towards females, of Bactrocera oleae flies aged from 3 to 6 days (ordinate), in the absence (without odor or solvent) or in the presence of a composition comprising, respectively, 2%, 4% and 5% propanoic acid (abscissa). In the figure, the numbers represent the number of pairs observed.

FIG. 19 is a bar chart showing the measurement of the proportion, in percentage, of courtship, of males towards females, of Bactrocera oleae flies aged from 13 to 19 days (ordinate), in the absence (without odor or solvent) or in the presence of a composition comprising, respectively, 2% and 5% propanoic acid (abscissa). In the figure, the numbers represent the number of pairs observed.

FIG. 20 is a bar chart showing the time (in minutes or in seconds) required for the male and female Drosophila melanogaster flies (abscissa) to wake up from the anesthesia (ordinate) caused by exposure to a composition comprising, respectively, 2% propanoic acid (left-hand side on the abscissa), or 2% butyric acid (right-hand side on the ordinate). In the figure, the numbers represent the number of individuals tested, respectively, 20 males and 30 females previously exposed to 2% propanoic acid, and 6 males and 7 females previously exposed to 2% butyric acid.

### EXAMPLES

### Example 1: Effect of Propionic Acid and/or Butyric Acid on the Reproduction of Brachycera

In this example, the brachycera used were the flies (muscina) Drosophila melanogaster and Drosophila suzukii, In this example, a study of the courtship and the copulation was carried out.

A) Material and Method

1. Evaluation of the Courtship

1.1. Drosophila melanogaster

Drosophilae (Drosophila melanogaster) aged from 3 to 9 days are used for the courtship tests as described Grosjean et al. Nat. Neurosci. 2008 [1] and Grosjean et al. Nature 2011 [2].

Naive males were isolated in tubes, individually, upon emerging from their puparium. The females were selected to be virgin and were stored in tubes of 10 individuals. These tubes were placed in incubators, for which a temperature of 25° C. was kept constant. A 12 hour/12 hour day/night cycle was applied. The courtship tests were carried out in the morning, between 1 and 3 hours after the start of the day cycle.

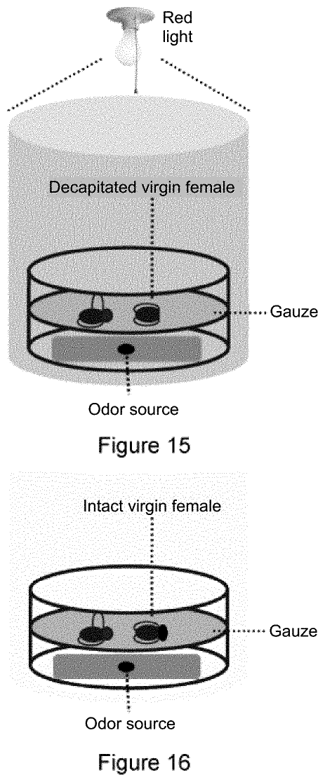

These tests were conducted in devices made up of 6 cylindrical, transparent, and closed alveolae (see FIG. 15) of a volume of 1.52 or cm³. A disk of filter paper (Whatman, filter paper 42) of 6 mm in diameter is deposited on the base thereof. Said paper is soaked in 10 μl of a composition comprising propionic acid and/or butyric acid diluted in paraffin oil (solvent). The different compositions used comprised, respectively, 0.5% or 1% propionic acid, 0.5% or 1% butyric acid. For each concentration, the number of pairs of flies tested was, respectively, 23, 23, 19 and 19. At the time of the test, 1 pair per alveolus was introduced into the behavior device (i.e. 6 pairs). Said pair was made up of one male and one female, each originating from the tubes mentioned above. The drosophilae were physically isolated from the odor source (said paper) by a netting which prevents any direct contact, the female had been decapitated but remained alive. In addition, the device was illuminated by red light (red far from the wavelength of between 625 and 630 nm) so as to eliminate any behavior associated with the visual perception which, in D. melanogaster, is between 300 and 550 nm)). Thus, the behavior observed depends only on the olfactory perception by the male of the compound introduced into the device at the time of the assembly thereof.

The behavior was filmed for 10 minutes, using a Sony HDR-XR550 camera. Each film was recorded and kept for subsequent analysis.

A proportion of courtship, expressed as a percentage, was then calculated, and represents the time during which the male courts the female during the 10 minutes of recording the test. The courtship of the Drosophila melanogaster is decomposed into recognizable stereotypical events, and described precisely in scientific literature (Greenspan & Ferveur, Annu. Rev. Genet, 2000 [3]). A statistical test (Mann-Whitney test (nonparametric test, GraphPad, Prism8)) was carried out in order to evaluate the influence, on the behavior, of the composition in which the paper is soaked; this involving a release of volatile compounds and an odor perceived by the drosophilae.

A negative control was carried out and corresponded to soaking the filter paper in a solution of paraffin oil alone (the solvent) in order to verify the absence of any effect on the flies.

1.2. Drosophila suzukii

Drosophilae (Drosophila melanogaster) aged from 3 to 9 days are used for the courtship tests according to the method described above.

Naive males were isolated in tubes, individually, upon emerging from their puparium.

The females were selected to be virgin and were stored in tubes of 10 individuals. These tubes were placed in an inclined manner in incubators, for which a temperature of 25° C. was kept constant. A 12 hour-12 hour day/night cycle was applied. The courtship tests were carried out in the morning, between 1 and 3 hours after the start of the day cycle.

At the time of the test, a 6 mm disk of filter paper (Whatman, filter paper 42) was deposited per alveolus of the device for studying the courtship behavior. Each alveolus was cylindrical, transparent and closed, and of a volume of 1.52 or 0.9 cm³. FIG. 16 is a schematic representation of an alveolus of the device used.

The paper was then soaked in 10 μl of a composition of 0.1%, 0.5% or 1% propionic acid, or 0.1%, 0.5% or 1% butyric acid, or a mixture comprising 0.1% propionic acid and 0.1% butyric acid, 0.25% propionic acid and 0.25% butyric acid or 0.5% propionic acid and 0.5% butyric acid. A gauze (netting) makes it possible to physically isolate the drosophilae from the odor. 6 pairs (one per alveolus) were observed independently. Each pair was made up of one male and one intact female, each originating from the tubes mentioned above.

The behavior was filmed for 10 minutes, under white light, using a Sony HDR-XR550 camera. Each film was recorded and then analyzed. For each concentration of the compositions used, the number of pairs of flies was, respectively, 33, 39, 27, 38, 33, 27, 12, 6 and 12.

A negative control was carried out comprising only the solvent, i.e. paraffin oil, in order to verify the absence of an effect of the solvent on the flies.

A courtship percentage was calculated, and represents the time during which the male courts the female during the 10 minutes of observation (recording). The courtship of the Drosophila suzukii is decomposed into recognizable stereotypical events, and described precisely in scientific literature (Revadi S et al., Insects 2015 [4]). Statistical tests, i.e. a Kruskal-Wallis test followed by a post-hoc Dunn's Multiple Comparison Test, were carried out (GraphFad, Prism6) in order to evaluate the influence, on the behavior, of the composition in which the paper is soaked; this involving a release of volatile compounds and an odor perceived by the drosophilae.

2. Evaluation of the Copulation

From the films recorded for studying the courtship behavior of Drosophila suzukii, a percentage of the number of pairs copulating during the 10 minutes of observation was calculated on the basis of the composition in which the filter paper was soaked. A statistical evaluation was carried out, and different tests were performed. In particular, a Kruskal-Wallis test followed by a post-hoc Dunn's Multiple Comparison Test were carried out (GraphPad, Prism6) in order to quantify the influence, on the number of copulating individuals, of the composition in which the paper is soaked and which involves a release of volatile compounds and an odor perceived.

B) Results

1. Evaluation of the Courtship

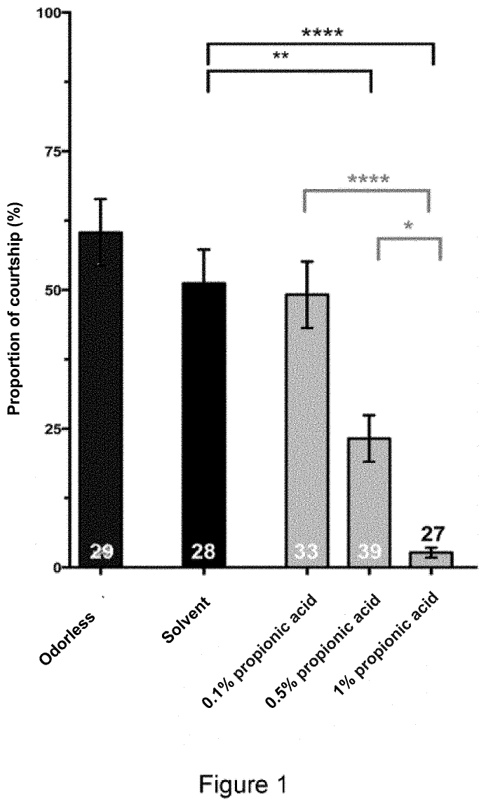

FIGS. 1 to 3 show the results obtained using Drosophila suzukii flies, depending, respectively, in particular on the presence or absence of propionic acid, butyric acid, or a mixture of propionic acid and butyric acid.

As shown in FIG. 1, in the presence of 0.5% propionic acid in the solvent the courtship was significantly reduced, compared with the solvent alone (**). In the presence of 1% propionic acid in the solvent, the courtship was significantly reduced compared with the solvent alone (****); likewise, compared with a composition comprising 1% (****) vs. 0.1%, or 1% vs. 0.5% propionic acid (*).

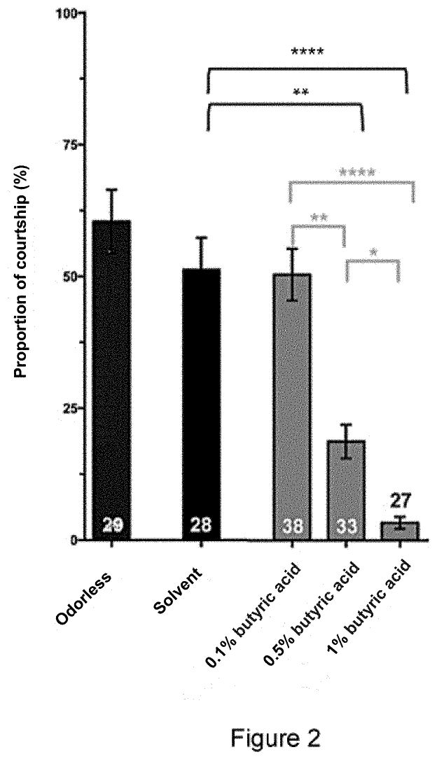

As shown in FIG. 2, in the presence of 0.5% butyric acid in the solvent the courtship is significantly reduced, compared with the solvent alone (**); likewise, compared with 0.1% butyric acid in the solvent (**).

In the presence of 1% butyric acid in the solvent the courtship is significantly reduced, compared with the solvent alone (****); likewise, compared with 0.1% (****) and 0.5% (*).

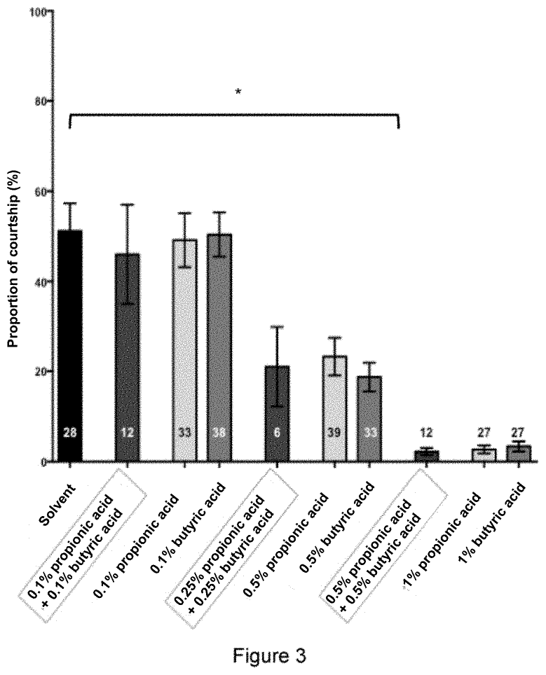

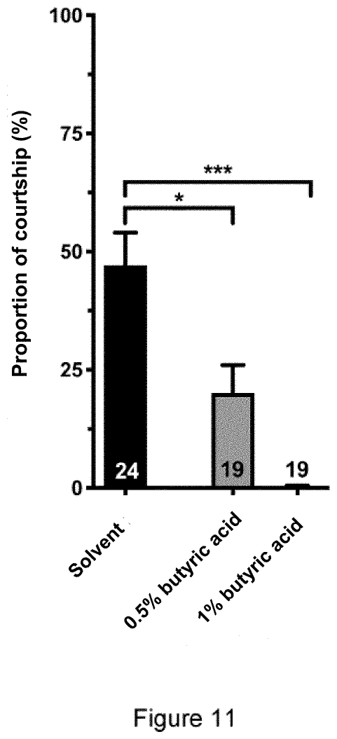

As shown in FIG. 3, in the presence of 0.25% propionic add and 0.25% butyric add in the solvent the courtship was reduced, compared with the solvent alone. In the presence of 0.5% propionic add and 0.5% butyric add in the solvent the courtship was significantly reduced, compared with 0.1% propionic acid and 0.1% butyric acid in the solvent (*), and with 0.5% propionic acid and 0.5% butyric acid in the solvent. FIGS. 10 and 11 show the results obtained using Drosophila melanogaster flies, depending, respectively, in particular on the presence or absence of propionic acid or butyric acid.

As shown in FIG. 10, the proportion of courtship of males towards females was significantly changed (*) in the presence of the composition comprising 1% propionic acid, compared with the solvent alone. The courtship was also significantly reduced (*) in the presence of 1% propionic acid in the solvent, compared with 0.5% propionic acid in the solvent.

As shown in FIG. 11, the proportion of courtship of males towards females was significantly reduced (*) in the presence of a composition comprising 0.5% butyric acid, compared with the courtship in the solvent alone. The courtship was also significantly reduced (***) in the presence of a composition comprising 1% butyric acid, compared with the courtship in the solvent alone.

2. Evaluation of the Copulation

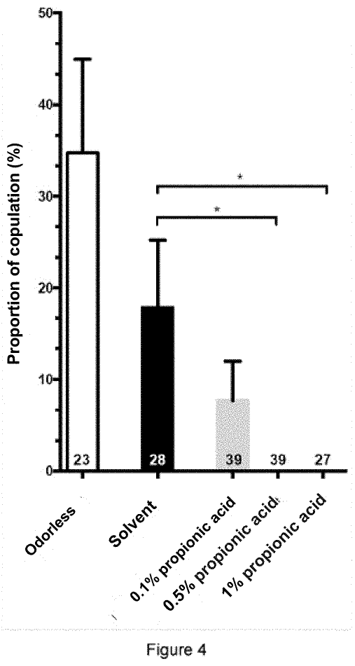

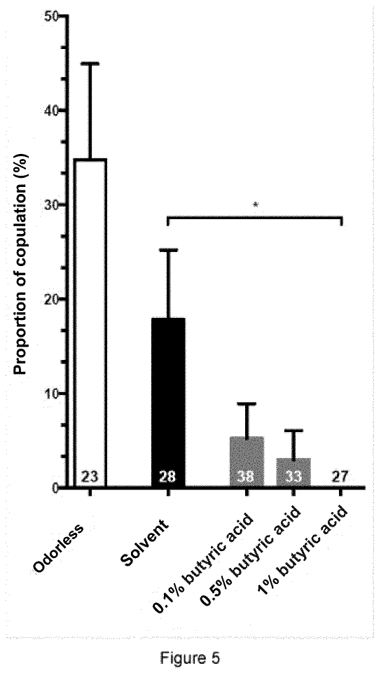

FIGS. 4 and 5 show the results obtained using Drosophila suzukii flies, depending, respectively, in particular on the presence or absence of propionic acid, butyric acid, or a mixture of propionic acid and butyric acid.

As shown in FIG. 4, the proportion of copulation was reduced by 58% in the presence of a composition comprising 0.1% propionic acid diluted in the solvent. In the presence of a composition comprising 0.5% propionic acid ((*) vs. solvent alone) or 1% propionic acid ((*) vs. solvent alone), the proportion of copulation is zero; no copulation was observed.

As shown in FIG. 5, the proportion of copulation was reduced by 70.55% in the presence of a composition comprising 0.1% butyric acid. In the presence of a composition comprising 0.5% butyric acid, the proportion of copulation was reduced by 83.03% (going from 17.86% to 3.03%), and in the presence of a composition comprising 1% butyric acid ((*) vs. solvent alone), the proportion of copulation is zero; no copulation was observed.

As shown above, the presence of butyric acid and/or propionic acid makes it possible to control the reproduction of brachycera. In particular, as shown above, examples of compositions according to the invention make it possible to both reduce the courtship of males towards females, and also to reduce or even suppress the copulation of brachycera.

This example also demonstrates that propionic acid, butyric acid and/or a derivative thereof are useful as an active principle for controlling the reproduction of brachycera.

This example moreover demonstrates that propionic acid, butyric acid and/or a derivative thereof are useful as an active principle for controlling the reproduction of brachycera at low concentrations, advantageously making it possible to obtain a rapid effect while reducing and/or avoiding any possible side effect associated, for example, with high concentrations.

### Example 2: Effect of Propionic Acid and/or Butyric Acid and/or a Derivative of Propionic Acid on the Reproduction of Brachycera and/or as Repellent Active Principles

In this example, the brachycera used were the flies Drosophila melanogaster and Drosophila suzukii. The drosophilae (Drosophila melanogaster and Drosophila suzukii) were as described in example 1, although the D. melanogaster females had not been decapitated.

In this example, a study of an anesthetic effect and of a repellent effect of propionic acid and/or butyric acid was carried out.

A) Material and Method

1. Anesthesia Tests:

The devices used were similar to the devices for studying the courtship (example 1 above), except for the volume of the space into which the files were introduced, which was 45.24 or 22.62 cm³. 9 discs of filter paper (Whatman, filter paper 42), each soaked in 10 μl of a composition comprising butyric acid and/or propionic acid and/or a derivative of propionic acid diluted in paraffin oil were deposited in the device. The different compositions used comprised, respectively, 2% propionic acid or 2% butyric acid or 5% ethyl propanoate or 10% ethyl propanoate or 30% ethyl propanoate. For each composition, the number of flies was, respectively, 30 and 24 males, 30 and 11 females for Drosophila melanogaster, and 24, 16 and 4 males, 28, 26, 6, 5 and 4 females for Drosophila suzukii. In order to avoid any contact of the flies with the soaked disks of paper, a gauze (netting) was arranged above said papers. The drosophilae could move freely on said gauze. The quantity of compound, butyric acid or propionic acid, or of ethyl propanoate (odor) volatilized in the behavior chamber was equivalent to that of the device used for the courtship. Likewise, the flies were never in direct contact with the composition (no taste perception), only with the volatile particles (olfactory perception). The flies tested were introduced simultaneously, by group, into the enclosure. Their behavior was filed for 10 minutes, and a time required for leading to anesthesia was calculated over this period, depending on the concentration of butyric acid or propionic acid or ethyl propanoate used. The flies were considered anesthetized when they stopped moving and fell on their side. A Mann-Whitney test (nonparametric test, GraphPad, Prism 6 or Prism 8) did not reveal any difference in the time elapsed before anesthesia for the males or the females tested.

2. Tests “in a Box” (Repellent Effect)

The sensitivity of the drosophilae was evaluated over a period of between 0 minutes and 24 hours, according to the conditions (see results) in closed and transparent boxes of 1250 cm³.

An odor source, i.e. a composition comprising butyric acid or propionic acid in paraffin oil, was deposited on a filter paper at the base of a 50 mm petri dish. The petri dish is introduced into a test box and covered with a gauze Securimed M8202 sterile gauze pad) in order to prevent the flies from getting stuck on the oily odorous compound.

The concentrations used were 2%, 6% or 20% propionic acid.

A second Petri dish containing 5 to 6 grams of synthetic food used for breeding drosophilae in laboratories was placed in the test box, i.e. 6.5% maize flour, 6.5% (v/v) yeast extract, 1% (v/v) agar-agar, and 3% (v/v) of a 0.1% (v/v) antifungal solution (Tegosept (Apex)) in ethanol at 95%. This latter provides the water and/or the food which the flies needed during the test, and eliminated the possibility of an event due to dehydration of the individuals tested.

Drosophila melanogaster or suzukii were then introduced by groups into the test box, and moved there freely over time. Their behavior in the presence of a composition as mentioned above was observed and photographed (Apple iPhone 6S).

B) Results

1. Anesthesia Tests.

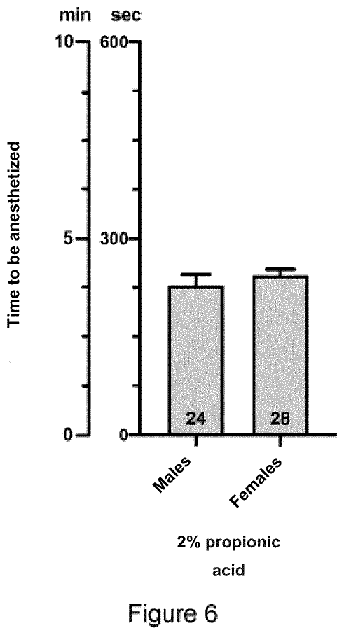

FIGS. 6 to 9 and FIG. 17 show the results obtained using Drosophila suzukii flies, depending, respectively, on the presence or absence of propionic acid or butyric acid or ethyl propanoate.

As shown in FIG. 6, in the presence of 2% propionic acid the male flies and the female flies were anesthetized in approximately 4 minutes. A statistical evaluation (Mann-Whitney test, p=0.7056) showed no significant difference in the time required for males or females to be anesthetized.

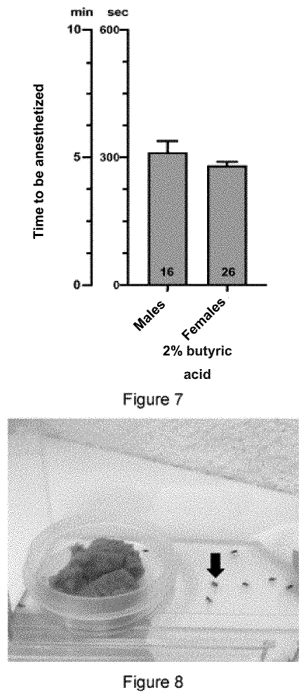

As shown in FIG. 7, in the presence of 2% butyric acid the male flies and the female flies were anesthetized in approximately 5 minutes. A statistical evaluation (Mann-Whitney test, p=0.43) showed no significant difference in the time required for males or females to be anesthetized.

FIG. 8 is a photograph showing the anesthesia of Drosophila suzukii flies following their introduction into a transparent test box of 1250 cm³, in which propionic acid diluted to 2% in the solvent has been deposited in a petri dish covered by a gauze in order to avoid any direct contact. The individuals first remained preferably away from said odor, and then the males and the females were anesthetized in the hour which followed. The photograph indicates the location of the flies and one individual on its back, in the anesthesia position, in the hour following its introduction into the box.

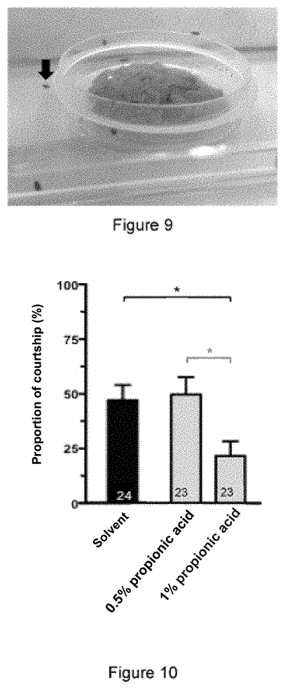

FIG. 9 is a photograph showing the anesthesia of Drosophila suzukii flies following their introduction into a transparent test box of 1250 cm³, in which butyric acid diluted to 2% in the solvent has been deposited in a petri dish covered by a gauze in order to avoid any direct contact. The flies present in the transparent test box remained away from the filter soaked in butyric acid, the males and females were anesthetized, and died after 20 hours in this environment. The photograph indicates the location of the flies and one individual on its back, in the anesthesia position, in the hour following its introduction into the box.

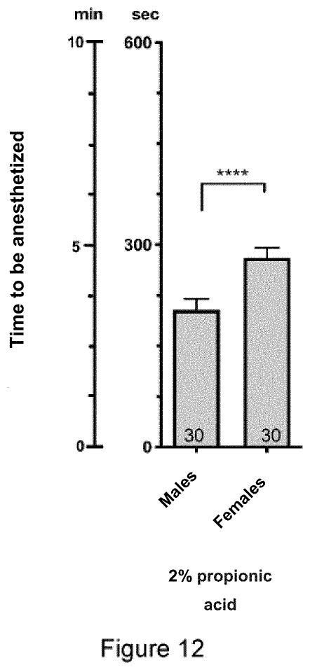

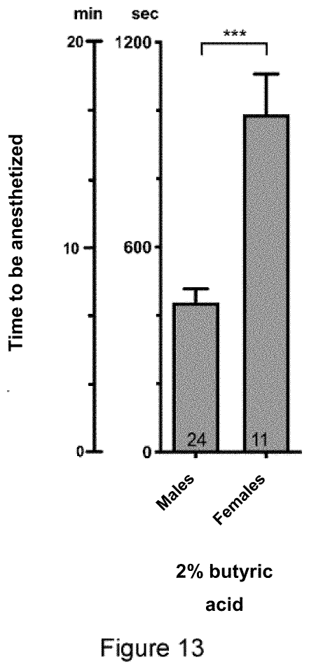

FIGS. 12 and 13, obtained in the devices described in A1 show the results obtained using Drosophila melanogaster flies, depending, respectively, in particular on the presence or absence of propionic acid or butyric acid.

As shown in FIG. 12, in the presence of 2% propionic acid the male flies were anesthetized in approximately 3 minutes, and the female flies were anesthetized in approximately 5 minutes. A statistical evaluation (nonparametric Mann-Whitney test) between the time required for the male or female flies to be anesthetized demonstrated a greater sensitivity of the males (****).

As shown in FIG. 13, in the presence of 2% butyric acid the male flies were anesthetized in approximately 5 minutes, and the female flies were anesthetized in approximately 17 minutes. A statistical evaluation (nonparametric Mann-Whitney) between the time required for the male or female flies to be anesthetized demonstrated a greater sensitivity of the males (****).

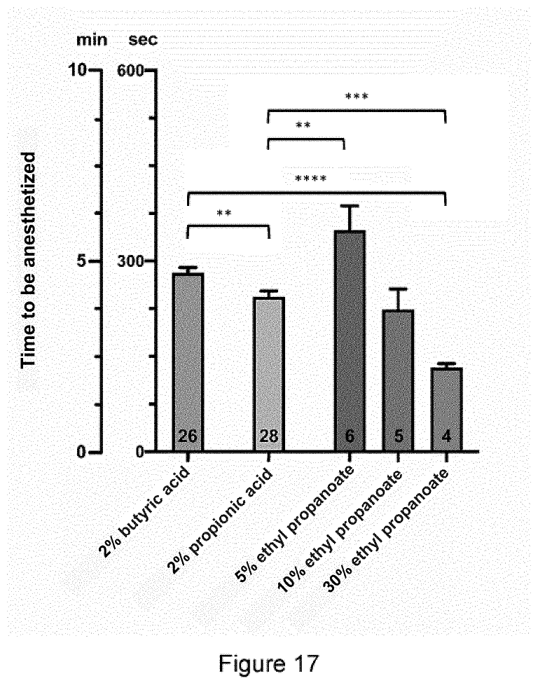

FIG. 17, obtained in the devices described in A1 shows the results obtained using female Drosophila suzukii flies in the presence of 2% butyric acid or 2% propionic acid or 5% ethyl propanoate or 10% ethyl propanoate or 30% ethyl propanoate. As shown in FIG. 17, in the presence of 2% butyric acid the female flies were anesthetized in approximately 5 minutes, and in the presence of 2% propionic acid the female flies were anesthetized in approximately 4 minutes. A statistical evaluation (nonparametric Mann-Whitney) of the time required for the females to be anesthetized shows an increased sensitivity (**) in the presence of 2% butyric acid in the solvent compared with 2% propionic acid in the solvent. In the presence of 5% ethyl propanoate the female flies were anesthetized in approximately 6 minutes, in the presence of 10% ethyl propanoate the female flies were anesthetized in approximately 4 minutes, which does not demonstrate any statistical difference with respect to 2% propionic acid and 2% butyric acid. Furthermore, in the presence of 30% ethyl propanoate the female flies were anesthetized in approximately 2-3 minutes, which corresponds to a significant statistical difference with respect to 2% butyric acid (****) and with respect to 2% propionic acid (***).

2. Tests of the Repellent Effect (Tests “in a Box”)

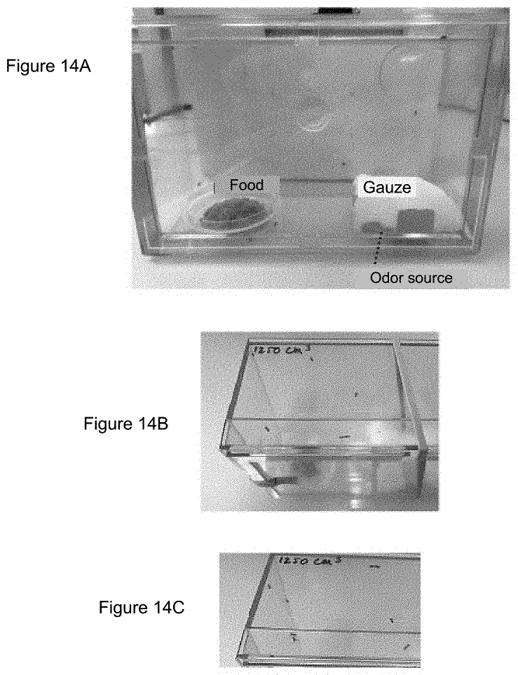

FIG. 14 shows the results obtained using Drosophila melanogaster flies. In a box containing propionic acid diluted to 2% or to 6% in the solvent (paraffin oil), the flies are still alive after 24 hours. During this period, males and females court and copulate (FIG. 14); removed from the box they will have viable offspring.

At 20% propionic acid diluted in the solvent, the individuals keep away from the filter paper soaked with the odorous composition, and then are anesthetized in the test hour.

### Example 3: Effect of Propionic Acid and/or Butyric Acid and/or a Derivative of Propionic Acid on the Reproduction of Brachycera and/or as Repellent Active Principles

In this example, the brachycera used were the flies Bactrocera oleae aged 3 to 6 days or 13 to 19 days, respectively.

A) Material and Method

1. Evaluation of the Courtship
Olive flies (Bactrocera oleae) aged from 3 to 6 days or sexually mature (from 9 to 13 days) were used for the tests of courtship of males towards females (Bonelli G., J. Insect Behay. 2012 [6] and Menage Cindy, personal communication). Naive males were isolated in tubes, individually, upon emerging from their puparium. The females were selected to be virgin and were stored in tubes, together. These tubes were placed in an inclined manner in incubators, for which a temperature of 25° C. was kept constant. A 12 hour/12 hour day/night cycle was applied. The courtship tests were carried out in the morning, 1 hour before the end of the day cycle, when the males are at their most active (Bonelli G., J. insect Behav. 2012 [6]).
At the time of the test, 3 disks of filter paper (Whatman, filter paper 42) of 6 mm in size were deposited per alveolus of the device for studying the courtship behavior. Each alveolus was cylindrical, transparent and closed, and of a volume of 5.9 cm³. FIG. 16 is a schematic representation of an alveolus of the device used.
The papers were then each soaked in 10 ml of a composition of 2%, 4% or 5% propionic acid. A gauze (netting) made it possible to physically isolate the B. ° lea from the odor corresponding to the propionic acid. 2 pairs (one per alveolus) were observed independently. Each pair was made up of one male and one female, each originating from the tubes mentioned above.
The behavior was filmed for 10 minutes, under white light, using a Basler acA1920-155 um USB 3.0 camera. Each film was recorded (Basler Video Recording Software V1.3) and then analyzed. For each concentration of the compositions used, the number of pairs of flies was, respectively, 2, 5, 2, 2, 2 and 6, 6, 4, 4.
A negative control was carried out comprising only the solvent, i.e. paraffin oil, in order to verify the absence of an effect of the solvent on the flies.

A courtship percentage was calculated, and represents the time during which the male courts the female during the 10 minutes of observation (recording). The different steps consist in movement of the wings (rapid “buzzing” and/or vertical vibrations), face movements, lateral movements, “dances” in rapid circles and in the direction of the female (Benelli, J. Insect Beahv 2012 [6] and Ménagé Cindy, personal communication).

B) Results.

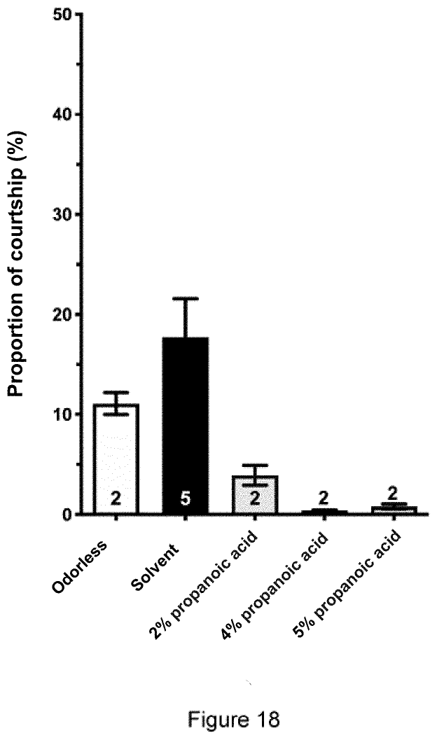

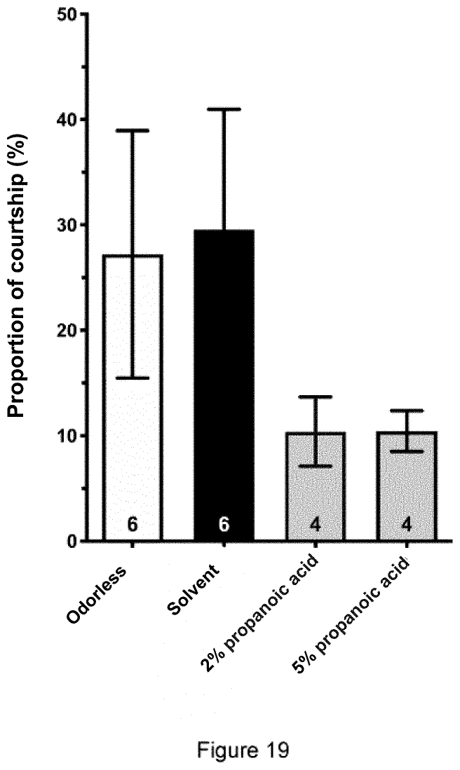

FIGS. 18 and 19 show the results obtained using Bactrocera plea flies, depending on the presence or absence of propionic acid and according to their age.

As shown in FIG. 18, in the presence of 2% propionic acid in the solvent the courtship of males towards females was significantly reduced, compared with the solvent alone. In the presence of 4% and 5% propionic acid in the solvent, the courtship was virtually nonexistent.

As shown in FIG. 19, in the presence of a composition comprising 2 or 5% propionic acid, in other words in the presence of 2% and 5% propionic acid in the solvent, the courtship of males towards females is significantly reduced, compared with the solvent alone.

As demonstrated above, the presence of propionic acid makes it possible to control the reproduction of brachycera. In particular, as demonstrated above, examples of compositions according to the invention make it possible to both reduce the courtship of males towards females.

This example also demonstrates that propionic acid is useful as an active principle for controlling the reproduction of brachycera without an insecticide effect.

Furthermore, this example clearly demonstrates that the presence of propionic acid advantageously allows for a reduction and/or inhibition of the courtship and/or copulation of brachycera. This example also clearly demonstrates that the presence of propionic acid a control of the reproduction prior to egg-laying.

This example moreover demonstrates that propionic acid is useful as an active principle for controlling the reproduction of brachycera at low concentrations, advantageously making it possible to obtain a rapid effect while reducing and/or avoiding any possible side effect associated, for example, with high concentrations.

### Example 4: Effect of the Suppression/Reversible Effect of Propionic Acid and/or Butyric Acid and/or a Derivative of Propionic Acid on the Anesthesia of Brachycera

In this example, the brachycera used were the flies Drosophila melanogaster. Said drosophilae (Drosophila melanogaster) were those described in example 2.

In this example, a study of a reversible and non-toxic effect of propanoic acid and/or butyric acid was carried out.

A) Material and Method

Test of the Reversible Effect.

The devices used were those described for the study of the anesthesia such as described in example 2 above. The application conditions for the propanoic acid or the butyric acid were similar to those of the anesthesia test described in example 2 above. The flies were brought into the presence of 2% propanoic acid or 2% butyric acid for 10 minutes. At the end of 10 minutes of observation of the anesthesia (example 2 above), the flies were removed from the device and placed into another device which was identical but “empty”, i.e. without solvent or odor (odor=2% propanoic acid in the solvent or 2% butyric acid in the solvent, respectively). For each empty device, the total number of flies introduced and observed were 20 males and 30 females previously exposed to 2% propanoic acid and removed from this anesthetizing environment, and 6 males and 7 females previously exposed to 2% butyric acid and removed from this anesthetizing environment.

The behavior of the flies in the empty devices was observed for 25 minutes and filmed.

The flies were considered as woken from the anesthesia when they got back up on their legs and moved in the device.

The time required for waking the flies following their anesthesia was then measured.

B) Results.

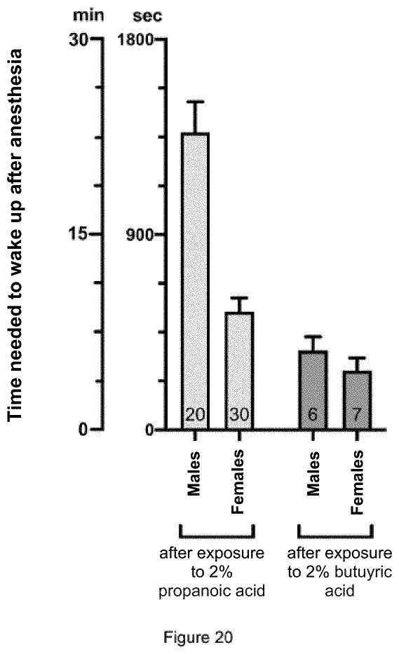

FIG. 20 shows the results obtained using Drosophila melanogaster flies, male and female respectively, following their exposure to 2% propanoic acid and/or to 2% butyric acid, respectively.

As shown in FIG. 20 (left-hand side of the X-axis), the males woke up fully from the anesthesia after 23 minutes when they had previously spent 10 minutes in the presence of 2% propanoic acid and had then been removed from this odorous device in order to place them in an empty device. The females woke up fully after 9 minutes when they had previously spent 10 minutes in the presence of 2% propanoic acid and had then been removed from this odorous device in order to place them in an empty device.

As shown in FIG. 20 (right-hand side of the X-axis), the males woke up fully from the anesthesia after 6 minutes when they had previously spent 10 minutes in the presence of 2% butyric acid and had then been removed from this odorous device in order to place them in an empty device. The females woke up fully after 4.5 minutes when they had previously spent 10 minutes in the presence of 2% butyric acid and had then been removed from this odorous device in order to place them in an empty device.

As demonstrated in this example, an example of a composition according to the invention advantageously allows for reversible anesthesia on the brachycera. Furthermore, this example clearly demonstrates that the use of an example of a composition according to the invention is, in particular on account of the reversible effect thereof, non-toxic with respect to brachycera.

### LIST OF REFERENCES

1. Grosjean, Y., Grillet, M., Augustin, H., Ferveur, J. F. and Featherstone, D. E. A glial amino-acid transporter Controls synapse strength and courtship in Drosophila. Nature Neuroscience 2008. Volume 11, pages 54-61. DOI: 10.1038/nn2019
2. Grosjean Y, Rytz R, Farine J P, Abuin L, Cortot J, Jefferis G S, Benton R. An olfactory receptor for food-derived odours promotes male courtship in Drosophila. Nature 2011. Volume 478(7368), pages 236-240. DOI:10.1038/nature10428.
3. Greenspan & Ferveur, Courtship in Drosophila. Annu. Rev. Genet. 2000. Volume 34, pages 205-232. DOI: 10.1146/annurev.genet.34.1.205
4. Revadi S, Lebreton S, Witzgall P, Anfora G, Dekker T and Becher PG. Sexual Behavior of Drosophila suzukii. Insects 2015. Volume 6(1), pages 183-196 (2015). DOI: 10.3390/insects6010183,
5. Remington Pharmaceutical Sciences, sixteenth edition, E. W. Martin (Mack Publishing Co., Easton, Pa., 1980).
6, Benelli G. Male Wing Vibration in the Mating Behavior of the Olive Fruit Fly Bactrocera olea (Rossi) (Diptera: Tephritidae). J. insect behav. 2012 Volume 25, pages 590-603. DOI: 10.1007/s10905-012-9325-9

1. Use of at least one fatty acid selected from the group comprising propionic acid and butyric acid as a repellent active principle and/or for controlling the reproduction of brachycera.

2. Use according to claim 1, wherein the brachycera are selected from the group comprising the insects of the family of the oestridae, the hippoboscidae, the nycteribiidae, the syrphidae, the tachinidae, the thaumatoxena, the thermitoxena, the trypetidae.

3. Use according to claim 1, wherein the brachycera are selected from the group comprising insects of the genus drosophila, glossina, calliphora, auchmeromyia, lucilia, sarcophaga, stomoxys.

4. Use according to claim 1, wherein the brachycera are selected from the group comprising Drosophila suzukii, Bactrocera oleae, preferably Drosophila suzukii.

5. Use according to claim 1, wherein the propionic acid is at a concentration of 0.1% to 30% by volume (v/v).

6. Use according to claim 1, wherein the butyric acid is at a concentration of 0.1% to 2% by volume (v/v).

7. Use according to claim 1, wherein the propionic acid and/or the butyric acid are in a composition further comprising an acceptable support.

8. Use according to claim 7, wherein the acceptable support is paraffin oil.

9. Use according to claim 1, wherein the fatty acid or a composition comprising the fatty acid and an acceptable support is applied by means of a diffuser, a spray, an atomizer, or an impregnated support.

10. Use according to claim 1, wherein the fatty acid or a composition comprising the fatty acid and an acceptable support is present in a case, the case further comprising a support comprising usage instructions.

11. Method for protecting at least one biotic and/or abiotic surface from brachycera, comprising the application, onto said surface, of at least one fatty acid as defined in claim 1 or a composition comprising the fatty acid and an acceptable support, wherein the abiotic surface is a surface of all or part of a vegetable and/or plant.
